(19) United States
(12) Patent Application Publication
(10) Pub. No.: US 2025/0171723 A1
(43) Pub. Date: May 29, 2025

(54) PASSIVE MICROFLUIDIC FLOW RECTIFIER

(71) Applicant: Government of the United States of America, as represented by the Secretary of Commerce, Gaithersburg, MD (US)

(72) Inventors: Mandy Brigitte Esch, Gaithersburg, MD (US); Longyi Chen, Gaithersburg, MD (US)

(21) Appl. No.: 18/798,038

(22) Filed: Aug. 8, 2024

Related U.S. Application Data
(60) Provisional application No. 63/610,558, filed on Dec. 15, 2023.
(60) Provisional application No. 63/603,802, filed on Nov. 29, 2023.

(51) Int. Cl. C12M 3/06; C12M 1/00; C12M 1/26

(52) U.S. Cl. CPC C12M 23/16; C12M 23/40; C12M 29/18; C12M 33/04

(57) ABSTRACT

A passive microfluidic flow rectifier includes a microfluidic cell culture channel, a first reservoir fluidly connected to and upstream of the cell culture channel; a second reservoir fluidly connected to and downstream of the cell culture channel; and a fluid passageway fluidly connecting the first reservoir and second reservoir. The microfluidic cell culture channel is configured to keep cell culture medium within the channel via capillary forces when either reservoir is empty, and the fluid passageway is configured to reload cell culture medium from the second reservoir to the first reservoir when the flow rectifier is tilted and or rotated, thereby creating substantially unidirectional recirculating fluidic flow of cell culture medium through the microfluidic cell culture channel from the first reservoir to the second reservoir.

### RELATED APPLICATIONS

This application claims the benefit of U.S. Provisional Patent Application Ser. No. 63/610,558 (filed Dec. 15, 2023) and U.S. Provisional Patent Application Ser. No. 63/603,802 (filed Nov. 29, 2023), both of which are herein incorporated by reference in their entireties.

### FEDERALLY-SPONSORED RESEARCH AND DEVELOPMENT

This invention was made with United States Government support from the National Institute of Standards and Technology (NIST), an agency of the United States Department of Commerce. The Government has certain rights in this invention.

### COPYRIGHT NOTICE

This patent disclosure may contain material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF INVENTION

The present invention relates generally to microfluidic flow rectifiers, and more particularly to a connected dual-reservoir, gravity-powered microfluidic flow rectifier.

### BACKGROUND

Body-on-a-chip devices could potentially predict drug efficacy and toxicity in humans better than animals. Better predictions of drug toxicity are needed, because currently only about 10% of drugs that show high efficacy and low toxicity in animal trials repeat that effect in trials with humans. The reasons for that discrepancy are not entirely known, but it is clear that animal physiology differs from human physiology, and that some metabolic pathways and enzymes are missing in animals. Multi-organ body-on-a-chip devices mimic the human body by providing a way to coculture several human organ mimics in physiologic ratios and re-circulate a blood surrogate among them. The most advanced devices to date already contain ten to fourteen human tissues.

### SUMMARY OF INVENTION

Despite those advances, body-on-a-chip devices have not yet been extensively used in industry. One of the most important obstacles is that the devices are not easy to use, and they require expensive pumps for their operation. One strategy to overcome this problem is to utilize gravity to drive liquid through the devices, and gravity-driven flow can be achieved by putting the device at an angle. Changing the angle periodically, however, will create bidirectional flow, which substantially limits the culture of one of the most important cell types in the device (endothelial cells), because they are sensitive to the direction of shear. There exist microfluidic circuits with elements that rectify the flow in such devices, but those designs require the addition of extra liquid to the device because they require a separate backflow channel. However, additional liquid will dilute drug metabolites and skew their toxicity profile (toxicity depends on concentration).

Herein is described reservoirs that contain a passive flow rectifier, and requires only small amounts of liquid to operate, because the invention eliminates the need for a liquid-filled backflow channel. Rather than becoming part of the fluidic circuit design, this flow rectifier can be added to any fluidic design to convert its fluidic flow from bidirectional to unidirectional when it is driven via gravity.

According to one aspect of the invention, a passive microfluidic flow rectifier includes a microfluidic cell culture channel, a first reservoir fluidly connected to and upstream of the cell culture channel; a second reservoir fluidly connected to and downstream of the cell culture channel; and a fluid passageway fluidly connecting the first reservoir and second reservoir. The microfluidic cell culture channel is configured to keep cell culture medium within the channel via capillary forces when either reservoir is empty, and the fluid passageway is configured to reload cell culture medium from the second reservoir to the first reservoir when the flow rectifier is tilted and or rotated, thereby creating substantially unidirectional recirculating fluidic flow of cell culture medium through the microfluidic cell culture channel from the first reservoir to the second reservoir.

Optionally, the microfluidic cell culture channel includes a capillary valve tending to prevent backflow.

Optionally, the microfluidic cell culture channel is small enough to hold cell culture medium within via capillary forces when either reservoir is empty.

Optionally, the microfluidic cell culture channel includes microscale ridges configured to produce capillary forces acting on the cell culture medium to prevent complete disconnection of fluid within the channel from fluid in at least one of the reservoirs.

Optionally, the fluid passageway is U-shaped.

Optionally, the fluid passageway is C-shaped.

Optionally, in a first orientation of the flow rectifier, the first reservoir is above the second reservoir, thereby creating a pressure difference tending to cause fluidic flow through the cell culture channel, and wherein in a second orientation of the flow rectifier, the two reservoirs are situated so as to cause flow through the fluid passageway from the second reservoir to the first reservoir.

Optionally, the passive microfluidic flow rectifier also includes one or more seeding ports at or adjacent to the microfluidic cell culture channel.

Optionally, the passive microfluidic flow rectifier includes a rocker or rotating device configured to change an orientation of the reservoirs with respect to gravity.

Optionally, the passive microfluidic flow rectifier includes one or more additional microfluidic cell culture channels and wherein the microfluidic cell culture channel and the additional microfluidic cell culture channels are in parallel with each other.

Optionally, the microfluidic cell culture channel includes one or more cell culture chambers along its length.

Optionally, the microfluidic cell culture channel is 3D printed.

Optionally, the microfluidic cell culture channel is made from a biocompatible material.

The foregoing and other features of the invention are hereinafter described in greater detail with reference to the accompanying drawings.

### BRIEF DESCRIPTION OF THE DRAWINGS

FIG. 1 shows an isometric view of an exemplary passive microfluidic flow rectifier with a U-shaped channel.

FIG. 2 shows a front view of an exemplary passive microfluidic flow rectifier with a U-shaped channel.

FIG. 3 shows a side view of an exemplary passive microfluidic flow rectifier with a U-shaped channel.

FIG. 4 shows a bottom view of an exemplary passive microfluidic flow rectifier with a U-shaped channel.

FIG. 5 shows a front view of an exemplary passive microfluidic flow rectifier with a C-shaped channel.

FIG. 6 shows a side view of an exemplary passive microfluidic flow rectifier with a C-shaped channel.

FIG. 7 shows exemplary microfluidic flow channel configurations.

### DETAILED DESCRIPTION

This application describes a passive microfluidic flow rectifier that can be used to create unidirectional microfluidic flow in microfluidic channels when they are tilted back and forth or rotated to create gravity-induced fluidic flow using gravity.

Exemplary embodiments can be used to create pumpless tissue-chips or body-on-chip systems. While other pumpless body-on-chip systems exist, typically their flow is bidirectional, which does not mimic the unidirectional flow in the human body. Bidirectional flow prohibits the inclusion of endothelial cells in pumpless tissue chips and pumpless body-on-a-chip systems, because endothelial cells decay under bidirectional flow. Yet, endothelial cells are the most important barrier a drug must cross in order to reach diseased tissues, especially in the brain. Since the primary application of tissue-chips and body-on-a-chip systems is to test drug uptake, efficacy, and toxicity, endothelial cells are desirable to include in order to make accurate measurements. Exemplary embodiments allow for the culture of endothelial cells within pumpless tissue chips and body-on-a-chip systems.

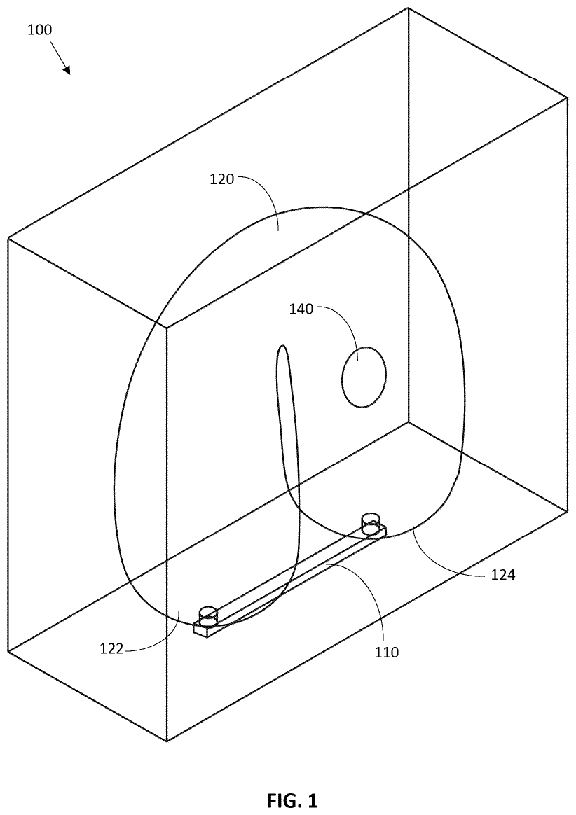

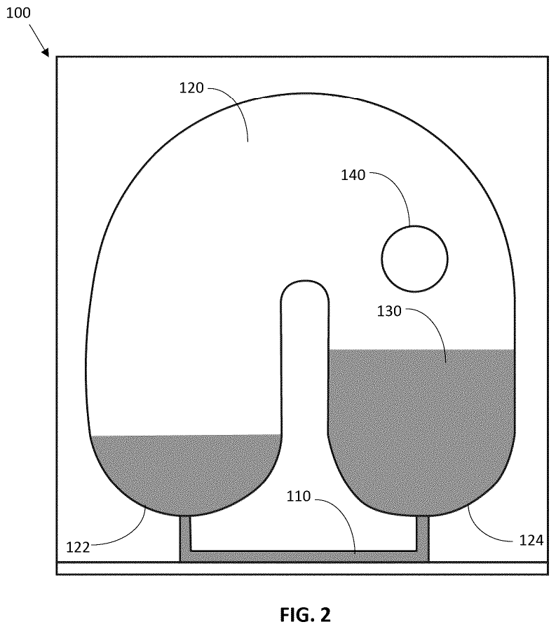

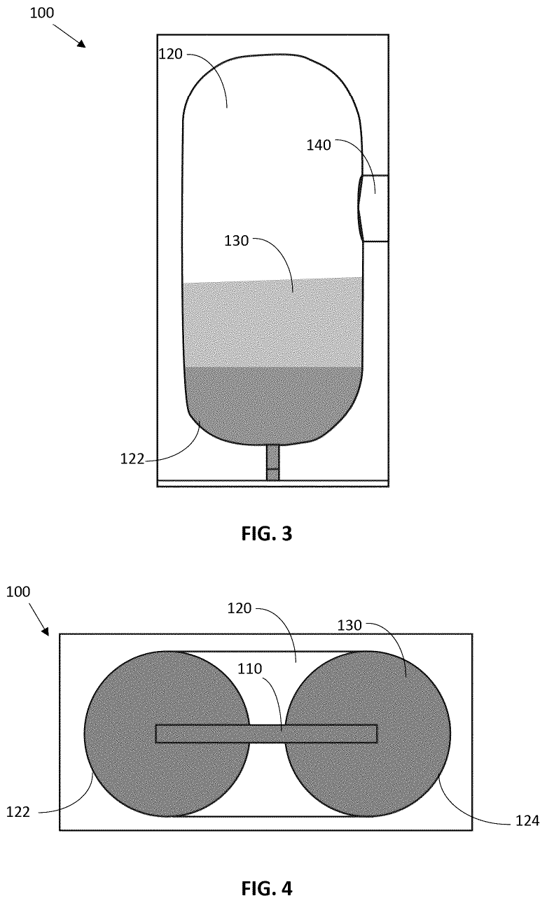

Referring to FIG. 1-4, an exemplary flow rectifier is shown at 100. The flow rectifier 100 includes a cell culture channel 110 and a U-shaped tube 120 with ends 122, 124. The two ends 122, 124 act as liquid reservoirs that, depending on the positioning of the device are situated at different heights, thereby creating a pressure difference that leads to fluidic flow through the cell culture channel 110. The U-shaped channel 120 is used to reload the cell culture medium 130 into the upper medium reservoir 124 once it has been depleted. The device 100 creates unidirectional recirculating fluidic flow of small amounts of cell culture medium 130 (50 μL to 200 μL). Medium 130 may be added or subtracted via port 140, which may be any appropriate port known in the art.

The height between the higher reservoir 124 and the lower reservoir 122 can be modulated in several ways to achieve different gravity pressure, and, therefore, different flow rates. For example, the device 100 can be designed with longer and/or narrower U-shaped path than that shown. For any given design, the amount of cell culture medium 130, and the microfluidic channel 110 dimensions (and with that the channel's hydraulic resistance) can be adjusted to produce the desired flow rate.

When the device 100 is held up at any certain angle in which the height of medium 130 in reservoir 124 is higher than the medium 130 of reservoir 122, medium will flow through the microfluidic channel 110 from reservoir 124 to reservoir 122 because of the differences in liquid height levels. Once the liquid levels in the reservoirs 122, 124 are equal, the entire device may be rotated counterclockwise such that the medium continues to flow through the channel 110. Once rotated for enough, fluid from both reservoirs will pool in the bend of the U-shaped channel 120. When the device is then tilted or rotated back to its original position, the medium will all or mostly pool in the reservoir 124, resulting in a height difference that drives liquid flow in the direction from reservoir 124 to reservoir 122 again. This process is repeated to achieve unidirectional flow inside the microchannel. Backflow in the unwanted direction is prevented because when the liquid is pooled in the bigger bend of the device, it is disconnected from both inlet and outlet. Additionally, capillary forces keep the liquid from leaving the microfluidic channel. The inlet microchannel may also be built with a micro extrusion to function as a capillary valve to stop backflow.

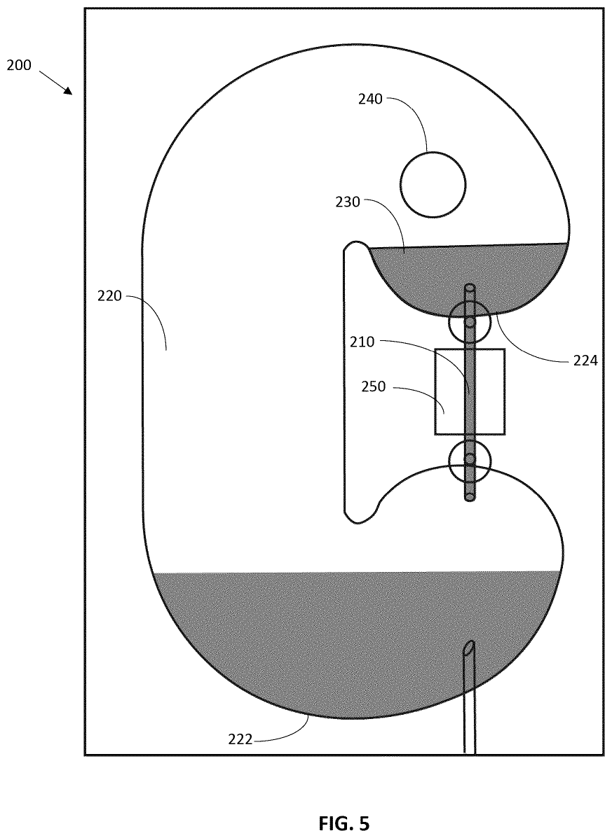

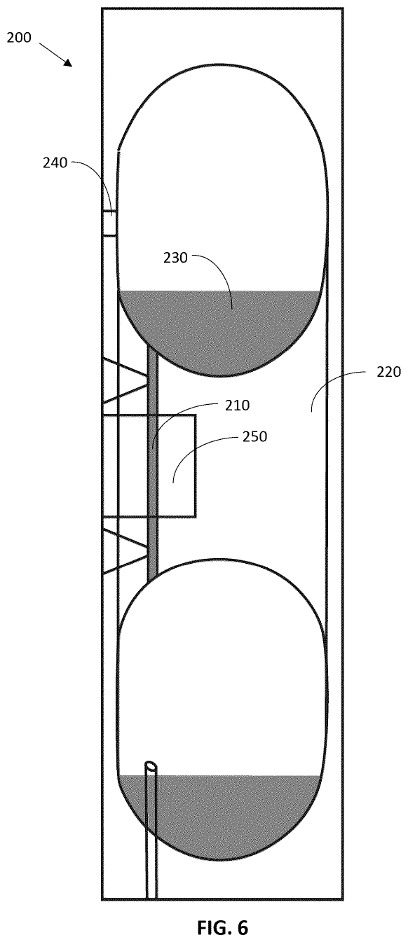

Turning now to FIG. 5-6, an exemplary embodiment of the flow rectifier is shown at 200. The flow rectifier 200 is substantially the same as the above-referenced flow rectifier 100, and consequently the same reference numerals but indexed by 100 are used to denote structures corresponding to similar structures in the flow rectifiers. In addition, the foregoing description of the flow rectifier 100 is equally applicable to the flow rectifier 200 except as noted below. Moreover, it will be appreciated upon reading and understanding the specification that aspects of the flow rectifiers may be substituted for one another or used in conjunction with one another where applicable.

Device 200 includes a more C-shaped channel 220 that holds the medium 230. The microfluidic channel 210 extends between ends 222, 224. An extracellular matrix (ECM) is shown at 250, with a microfluidic cell culture channel 210 flowing therethrough.

The C-shaped channel allows fluid to collect in the end 224 such that all the medium 230 may flow out of end 224 through the channel 210 while the device 200 is in that “upright” position shown. In other words, at steady-state, no fluid medium 230 would remain in the upper reservoir 224 with a C-shaped channel in the “upright” position. In contrast, with device 100, the upside-down U-shape results in medium 130 reaching an equilibrium between the two reservoirs 122, 124 at steady-state.

Exemplary devices may be placed on rocker or rotating platforms and may be operated under various conditions. For example, continuous rotation (for example, at a speed of three rotations per minute) at varying platform angles may be used. Alternatively, a device may be discontinuously rotated once) (360°) every 90 s. Alternatively, a device may be continuously rocked back and forth at angles between, e.g. 18° to −18°. Alternatively, a device may be discontinuously rocked where the device sits at an angle of, e.g., 18° for 90 s and at −18° for 2 s. In this manner, the shorter 2 s cycle is used as a reloading cycle that reloads the medium back into the top reservoir in a short amount of time.

The devices may be rotated while also being tilted off plane by some amount (e.g., 10° or 45°). This tilting may potentially improve elimination of backflow through the microfluidic channel and may be used to modulate the flow rate therethrough, where higher tilting angles will result in higher flow rates and lower tilting angles will result in lower flow rates.

Device designs with overall device dimensions of about 20 mm×32 mm×10 mm may be preferable. Such devices may be manufactured according to techniques known in the art, including 3D-printing, e.g. To render the devices biocompatible, they may be coated with a 6 μm to 10 μm thick layer of parylene C‡ using a vaporization chamber, e.g.

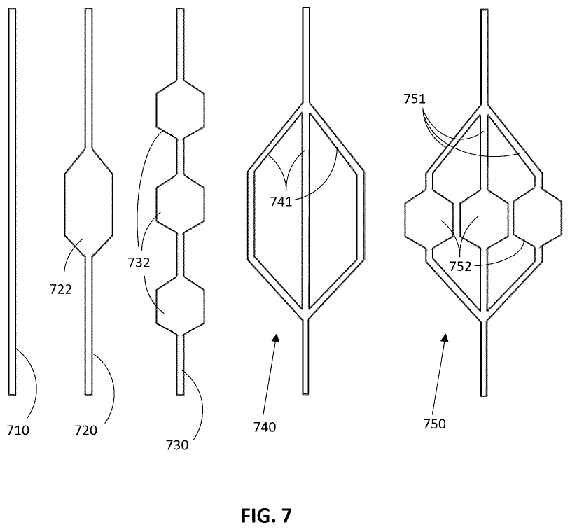

Exemplary embodiments may include a single cell culture channel, as illustrated and described above, or one or more channels in various configurations. Referring now to FIG. 7, illustrated are alternative cell culture channel arrangements. A single microfluidic cell culture channel 710 may be entirely uniforms and straight-walled. Alternatively, a single microfluidic cell culture channel 720 may include a cell culture chamber 722, performing as a single-organ microphysiological device. A single microfluidic cell culture channel 730 may include a plurality of cell culture chambers 732, performing as a multi-organ microphysiological device. Alternatively, a network of cell culture channels 740 may include a plurality of individual cell culture channels 741. The precise number of channels in the network may be determined by the application and is not limited to the network of three used as illustration. The plurality of channels 741 may be symmetric and uniform as illustrated, but may also include variations in size, shape, length, and other attributes as desired for the application. A network of channels 750 may also include channels 751 with one or more cell culture chambers 752, acting as a multi-organ microphysiological device. Although the network 750 is illustrated with chambers 752 in parallel configuration, and consistently on each channel 751, alternative arrangements may include one or more of the channels additionally having a plurality of chambers in series along a single channel, and, additionally or alternatively, may include one or more channels without any chambers.

The microfluidic cell culture channel may be 3D printed and closed with a glass cover slip that may be glued on using biocompatible adhesive. Such a channel may be, for example, approximately 10 mm long, 0.5 mm wide, and 1 mm high. This design enables imaging cultured cells using a microscope and fluorescence staining.

Another exemplary device may include a microfluidic channel approximately 200 μm in diameter.

Another exemplary device may include a microfluidic channel made from polymerizing collagen mixture around a needle (e.g. a 26 G needle).

Exemplary embodiments include a fluidic cell culture channel connecting two reservoirs (as well as any connecting channel segments) that is small enough to hold cell culture medium within via capillary forces when either reservoir is empty. Additionally, cell culture medium inside the reservoirs completely disconnects from the medium inside the microfluidic channels when the device is rotated or tilted. Additionally, cell culture medium inside the reservoirs reconnects with the medium inside the microfluidic channels at least on an upstream side once the rotation or tilting motion is completed.

3D printers often construct devices layer by layer. When multiple layers produce a rounded surface such as that of the u-shaped reservoir presented herein, the surface might contain microscale ridges. Those microscale ridges can produce capillary forces that act on the cell culture medium, and that prevent the complete disconnection of fluid in the u-shaped reservoir from the fluid inside the microfluidic channel. When such capillary forces are present, the fluidic flow in the devices may result in brief periods of negative, reverse flow (backflow). To achieve complete unidirectional flow, internal surfaces of the device should be as smooth as possible. Using higher resolution printers or other fabrication methods that deliver smoother prints are best suited to achieve that.

Other variables that affect the fluidic flow in the devices are the size of the microfluidic channel and the amount of medium that is used. A moment of reverse flow may occur when disconnection of the liquid in the channel from that in the u-shaped reservoir is delayed because of some medium that remained in the top reservoir. This condition occurs, when a small channel restricts fluidic flow to values that prevent the entire amount of medium to flow through the channel in any given cycle, or if the amount of medium in the top reservoir is too large to flow through the channel during the allotted time interval. If this is the case, either the channel size or the amount of cell culture medium can be adjusted to optimize the flow. Alternatively, the cycle time can also be lengthened to allow for more medium to pass through the channel.

Exemplary embodiments can be adjusted to accommodate longer cell culture channels as well as larger cell culture chambers as long as the main design principles are maintained. Longer channels and larger cell culture chambers can be accommodated by elongating the u-shaped reservoir. Elongating that reservoir can also accommodate larger amounts of cell culture medium should that be needed.

However, there is a lower size limit that can be achieved because cell culture medium can, in small reservoirs, create air bubbles when the medium is moved. The presence of air bubbles can disturb the process of reservoir medium disconnecting from the medium in the channel and with that interrupt the unidirectional flow pattern.

Exemplary devices may include a relatively large access hole (3 mm in diameter) but can also be designed with smaller cell seeding ports at the beginning and at the end of the microfluidic channels. Such designs may make cell seeding more reliable when a consistent cell seeding density is desired.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix (s) as used herein is intended to include both the singular and the plural of the term that it modifies, thereby including at least one of that term (e.g., the colorant(s) includes at least one colorants). Option, optional, or optionally means that the subsequently described event or circumstance can or cannot occur, and that the description includes instances where the event occurs and instances where it does not. As used herein, combination is inclusive of blends, mixtures, alloys, reaction products, collection of elements, and the like.

As used herein, a combination thereof refers to a combination comprising at least one of the named constituents, components, compounds, or elements, optionally together with one or more of the same class of constituents, components, compounds, or elements.

All references are incorporated herein by reference.

The use of the terms “a,” “an,” and “the” and similar referents in the context of describing the invention (especially in the context of the following claims) are to be construed to cover both the singular and the plural, unless otherwise indicated herein or clearly contradicted by context. It can further be noted that the terms first, second, primary, secondary, and the like herein do not denote any order, quantity, or importance, but rather are used to distinguish one element from another. It will also be understood that, although the terms first, second, etc. are, in some instances, used herein to describe various elements, these elements should not be limited by these terms. For example, a first current could be termed a second current, and, similarly, a second current could be termed a first current, without departing from the scope of the various described embodiments. The first current and the second current are both currents, but they are not the same condition unless explicitly stated as such.

The modifier about used in connection with a quantity is inclusive of the stated value and has the meaning dictated by the context (e.g., it includes the degree of error associated with measurement of the particular quantity). The conjunction or is used to link objects of a list or alternatives and is not disjunctive; rather the elements can be used separately or can be combined together under appropriate circumstances.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of 5 the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A passive microfluidic flow rectifier comprises:
a microfluidic cell culture channel;
a first reservoir fluidly connected to and upstream of the cell culture channel;
a second reservoir fluidly connected to and downstream of the cell culture channel; and
a fluid passageway fluidly connecting the first reservoir and second reservoir;
wherein the microfluidic cell culture channel is configured to keep cell culture medium within the channel via capillary forces when either reservoir is empty, and wherein the connection between either reservoir and the microfluidic channel is configured so that the liquid in the reservoir completely disconnects from the liquid within the channel when the device is tilted and/or rotated, and wherein the fluid passageway is configured to reload cell culture medium from the second reservoir to the first reservoir when the flow rectifier is tilted and or rotated, thereby creating substantially unidirectional recirculating fluidic flow of cell culture medium through the microfluidic cell culture channel from the first reservoir to the second reservoir.

2. The passive microfluidic flow rectifier of claim 1, wherein the microfluidic cell culture channel includes a structure tending to prevent backflow by promoting the disconnection of the liquid in the reservoir from the liquid in the channel when the device moves.

3. The passive microfluidic flow rectifier of claim 1, wherein the microfluidic cell culture channel is small enough to hold cell culture medium within via capillary forces when either reservoir is empty.

4. The passive microfluidic flow rectifier of claim 1, wherein the microfluidic cell culture channel includes surfaces that are free of microscale ridges and configured to prevent capillary forces acting on the cell culture medium to promote complete disconnection of fluid within the channel from fluid in at least one of the reservoirs.

5. The passive microfluidic flow rectifier of claim 1, wherein the fluid passageway is U-shaped.

6. The passive microfluidic flow rectifier of claim 1, wherein the fluid passageway is C-shaped.

7. The passive microfluidic flow rectifier of claim 1, wherein in a first orientation of the flow rectifier, the first reservoir is above the second reservoir, thereby creating a pressure difference tending to cause fluidic flow through the cell culture channel, and wherein in a second orientation of the flow rectifier, the two reservoirs are situated so as to cause flow through the fluid passageway from the second reservoir to the first reservoir.

8. The passive microfluidic flow rectifier of claim 1, further comprising one or more seeding ports at or adjacent to the microfluidic cell culture channel.

9. The passive microfluidic flow rectifier of claim 1, further including a rocker or rotating device configured to change an orientation of the reservoirs with respect to gravity.

10. The passive microfluidic flow rectifier of claim 1, further including one or more additional microfluidic cell culture channels and wherein the microfluidic cell culture channel and the additional microfluidic cell culture channels are in parallel with each other.

11. The passive microfluidic flow rectifier of claim 1, wherein the microfluidic cell culture channel includes one or more cell culture chambers along its length.

12. The passive microfluidic flow rectifier of claim 1, wherein the microfluidic cell culture channel is 3D printed.

13. The passive microfluidic flow rectifier of claim 1, wherein the microfluidic cell culture channel is injection-molded.

14. The passive microfluidic flow rectifier of claim 1, wherein the microfluidic cell culture channel is made from a biocompatible material.
